(19) United States
(12) Patent Application Publication
(10) Pub. No.: US 2009/0081297 A1
(43) Pub. Date: Mar. 26, 2009

(54) Use of surface tension reducing agents in aerosol formulations

(76) Inventors: Robert O. Cook, San Mateo, CA (US); Thomas A. Armer, Cupertino, CA (US)

Correspondence Address: MORRISON & FOERSTER LLP, 755 PAGE MILL RD, PALO ALTO, CA, 94304-1018, US

(21) Appl. No.: 11/412,523

(22) Filed: Apr. 27, 2006

Related U.S. Application Data
(60) Provisional application No. 60/675,375, filed on Apr. 27, 2005.

(51) Int. Cl. A61K 9/14; C07J 5/00

(52) U.S. Cl. 424/489; 552/577

(57) ABSTRACT

The present disclosure describes aerosol formulations that are particularly effective for pulmonary aerosol delivery. The aerosol formulations comprise an aqueous dispersion of active agent particles, said aqueous dispersion having an excess of a surface tension reducing agent. As a result of the reduced surface tension of the aqueous dispersion, the resulting aerosol droplets formed have a particle size less in one embodiment of than 10 microns in size or in an alternate embodiment of less than 6 microns in size. The present disclosure also provides for a method for forming an aerosol from said aerosol formulation, a method of treating a mammal in need of said treatment using said aerosol formulation, and a method of diagnosing a mammal in need of such diagnosis using said aerosol formulation.

This application claims priority to and benefit of U.S. Provisional patent application no. 60/765,375, filed Apr. 27, 2006.

### FIELD OF THE DISCLOSURE

The present disclosure is related to the field of drug delivery, particularly to improved aerosol formulations containing at least one active agent.

### BACKGROUND OF THE INVENTION

The delivery of drugs and other therapeutic agents to the respiratory tract is widely used for the treatment of a variety of diseases and conditions. Respiratory delivery is accomplished using an aerosol comprising drug particles surrounded by a liquid (referred to as a droplet) using a pressurized metered dose inhaler or nebulizer, or via the delivery of fine dry powders via a dry powder inhaler. The delivery of active agents to the respiratory tract offers several advantages over non-respiratory delivery. These advantages include, but not limited to, avoidance of metabolism via the first pass metabolic mechanisms and an increased efficiency of delivery to respiratory tissues (as compared to traditional administration via the bloodstream). However, delivery of drugs via the respiratory tract is critically dependent on the size of the droplets delivered to the respiratory tract or the size of the particles comprising the dry powder.

Using prior art delivery techniques, only approximately 10 to 30% of the packaged drug dosage is actually delivered to the lower respiratory tract. Specifically, there is loss due to the device used to deliver the active agent, loss of the active agent in the oral cavity and throat and with exhalation. These losses lead to variability in levels of the active agent and poor therapeutic control. In addition, deposition of the active agent to the mouth and throat can lead to systemic absorption and undesirable side effects.

The percentage of drug particles delivered to the respiratory tract, as discussed above, is directly related to the size of the droplets in the aerosol. Droplets having a size greater than about 10 microns do not enter the respiratory tract and are captured on the mucosal lining and the back part of the throat. Droplets in the size range of 1 to 10 microns are capable of entering the upper regions of the lung and greater than 60% of the droplets within this size range are deposited in the upper regions of the lung. Essentially 100% of droplets less than about 1 micron in size enter the lungs and pass into the peripheral alveolar region (the deep lung); however, about 50%-70% of droplets in this size range are exhaled.

Delivery to the deep lung also offers advantages in absorption of the active agent once deposited. Droplets deposited in the upper respiratory region are often cleared by the normal mechanisms of the lungs before significant drug absorption can occur. Cells in the upper respiratory tract utilize ciliated cells of the mucociliary escalator to remove foreign substance, such as the deposited droplets and the particles of active agent contained therein. As a result, the particles become trapped in the mucous coating the lung surface and are transported to the throat for removal by swallowing or coughing. However, such ciliated cells are absent in the deep lung, which relies on phagocytic mechanisms for clearance. Such phagocytic mechanisms operate more slowly than the ciliated cells in the upper respiratory tract, meaning droplets deposited in the deep lung have more time for absorption of the active agent, leading to increased delivery of the active agent to the subject. Furthermore, the peripheral alveoli have an increased surface area compared to regions of the upper respiratory tract meaning that absorption of the active agent occurs more rapidly.

In order to deliver the droplets deeper into the respiratory system, droplet size must be reduced. However, methods to consistently reduce droplet size are not well described in the art. The present disclosure provides a novel method to produce an aerosol composition comprising droplets of a size that can be delivered efficiently to the deep lung. The aerosol composition comprises droplets comprising an aqueous dispersion of at least active agent particles, preferably in nanoparticulate form, said aqueous dispersion having an excess of a surface tension reducing agent.

### DETAILED DESCRIPTION

The present disclosure provides an aerosol formulation, said aerosol formulation comprising an aqueous dispersion of active agent particles, said aqueous dispersion having an excess of a surface tension reducing agent. The active agent particles may be in nanoparticulate form. The active agent may be a therapeutic agent or a diagnostic agent and is insoluble or poorly soluble in water. As a result of the reduced surface tension of the aqueous dispersion, the resulting aerosol droplets formed have a particle size less in one embodiment of than 10 microns in size or in an alternate embodiment of less than 6 microns in size.

The present disclosure also provides for a method for forming an aerosol from said aerosol formulation, said aerosol formulation comprising droplets of an aqueous dispersion of active agent particles, said aqueous dispersion having an excess of a surface tension reducing agent. The active agent particles may be in nanoparticulate form. The active agent may be a therapeutic agent or a diagnostic agent and is insoluble or poorly soluble in water. In one embodiment, the method comprises the steps of:
a) providing an aerosol formulation comprising an aqueous dispersion of active agent particles, said aqueous dispersion having an excess of a surface tension reducing agent;
b) nebulizing or atomizing said aerosol formulation using an appropriate device to form an aerosol comprising droplets of said aerosol formulation, said droplets comprising particles of said at least one active agent and an excess of the surface tension reducing agent.

The present disclosure further provides a method of treating a mammal in need of said treatment, said method comprising the steps of:
a) providing an aerosol formulation comprising an aqueous dispersion of active therapeutic agent particles, said aqueous dispersion having an excess of a surface tension reducing agent;
b) nebulizing or atomizing said aerosol formulation using an appropriate device to form an aerosol comprising droplets of said aerosol formulation, said droplets comprising particles of said at least one active therapeutic agent and an excess of the surface tension reducing agent; and
c) administering said aerosol to the respiratory system of said mammal.

The present disclosure still further provides a method of diagnostic imaging of a mammal in need of such diagnosis, said method comprising:
a) providing an aerosol formulation comprising an aqueous dispersion of active diagnostic agent particles, said aqueous dispersion having an excess of a surface tension reducing agent;
b) nebulizing or atomizing said aerosol formulation using an appropriate device to form an aerosol comprising droplets of said aerosol formulation, said droplets comprising particles of said at least one active diagnostic agent and an excess of the surface tension reducing agent;
c) administering said aerosol to the respiratory system of said mammal; and
d) imaging said active diagnostic agent in said respiratory system.

An aerosol formulation according to the present disclosure for respiratory delivery generally comprises an aqueous dispersion of active agent particles, said aqueous dispersion having an excess of a surface tension reducing agent. The active agent particles may be in nanoparticulate form. As a result of the reduced surface tension of the aqueous dispersion, the resulting aerosol droplets formed from the aerosol formulation have a particle size less in one embodiment of than 10 microns or in an alternate embodiment of less than 6 microns. In one embodiment, the active agent particles are present in a nanoparticulate form. The active agent may be a therapeutic agent or a diagnostic agent. In one embodiment, the active agent is a non-water soluble or poorly water soluble in an aqueous media. By “poorly soluble” it is meant that the active agent has solubility in the aqueous media of less than about 10 mg/mL, or less than about 1 mg/mL. The aqueous media may be water or may be other media such as, but not limited to, aqueous salt solutions, safflower oil and solvents such as ethanol, t-butanol, hexane and glycol. The pH of the aerosol formulation can be adjusted by techniques known in the art. The aerosol formulation may comprise one or more than one active agents.

Exemplary therapeutic agents include, but are not limited to, a drug, a pharmaceutically active compound, a medicament, a peptide, and a nucleic acid. In one embodiment, the therapeutic agents for use in the present disclosure, include, but not limited to, analgesics, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, contrast media, corticosteroids, antibodies, cough suppressants (expectorants and mucolytics), diuretics, dopaminergics (antiparkinsonian agents), haemostatics, immunological agents, lipid regulating agents, muscle relaxants, parasympathomimetics, parathyroid calcitonin and biphosphonates, prostaglandins, sex hormones (including steroids), anti-allergic agents, stimulants and anoretics, sympathomimetics, thyroid agents, vasodilators and xanthines.

In a further embodiment, the therapeutic agent is a corticosteroid. As used herein, the term “corticosteroid” includes both mineral corticoids and glucocorticoids. The definition of corticosteroid is means to be broadly inclusive and is meant to include any compounds classified as a corticosteroid may be used, whether naturally occurring or synthetically prepared. The corticosteroid compounds may be in pure isomeric forms or mixed isomeric forms. In addition the corticosteroid compounds may be in the form of their esters, acetals, salts or other known forms. Examples of certain glucocorticoids include, but are not limited to, budesonide, dexamethasone, cortisone, prednisone, methylprednisone, hydrocortisone, beclomethasone dipropionate, betamethasone, flunisolide, fluticasone, flumethasone, fludrocortisone, diflorasone diacetate, flunisolide, fluocinolone acetonide, fluocinonide, fluorometholone, flurandrenolide, fluprednisolone, methylprednisone, paramethasone, prednisone, prednisolone, triamcinolone, alclometasone, amcinonide, cortisone, tetrahydrocortisol, clobetasol, ciclesonide, desonide, desiximetasonedeflazacort, halcinonide, medrysone, mometasone, paramethasone, tipredane, triamcinolone, and rofleponide. In one particular embodiment, the glucocorticoid compound is budesonide. Examples or mineral corticoids include, but are not limited to, aldosterone, fludrocortisone, and desoxycortiscosterone acetate. In a specific embodiment, the therapeutic agent is budesonide.

Exemplary diagnostic agents include, but are not limited to, diagnostic imaging agents, contrast agents, radio-pharmaceuticals and antibodies. Imaging agents include, but are not limited to, the x-ray imaging agent WIN-8883 (ethyl 3,5-diacetamido-2,4,6triiodobenzoate), WIN 67722 (6-ethoxy-6-oxohexyl-3,5-bis(acetamido)-2,4,6-triiodobenzoate), WIN 16318 (ethyl-2-(3,5-bis(acetamido)-2,4,6-triiodobenzoyloxy)butyrate), WIN 12901 (ethyl diatrizoxyacetate), WIN 16923 (ethyl 2-(3,5bis(acetamido)-2,4,6-triiodobenzoyloxy)propionate), WIN 65312 (N-ethyl 2-(3,5-bis(acetamido)-2,4,6triiodobenzoyloxy acetamide), WIN 12855 (isopropyl 2(3,5-bis(acetamido)-2,4,6-triiodobenzoyloxy)acetamide), WIN 67721 (diethyl 2-(3,5-bis(acetamido)-2,4,6triiodobenzoyloxy malonate); WIN 67585 (ethyl 2-(3,5bis(acetamido)-2,4,6-triiodobenzoyloxy)phenylacetate); propanedioic acid, WIN 68165 ([[3,5-bis(acetylamino)2,4,5-triodobenzoyl]oxy]-,bis(1-methyl)ester), WIN 68209 (3,5-bis(acetylamino)-2,4,6triodo-,4-(ethyl-3-ethoxy-2-butenoate)ester) and benzoic acid. Additional diagnostic agents suitable for use in the present disclosure are disclosed in U.S. Pat. Nos. 5,260,478; 5,264,610; 5,322,679 and 5,300,739.

Preferred diagnostic agents include those which are expected to disintegrate relatively rapidly under physiological conditions, thus minimizing any inflammatory response associated with the diagnostic agents. Therefore, in one embodiment the diagnostic agents are poorly soluble iodinated carboxylic acids such as iodipamide, diatrizoic acid, and metrizoic acid, along with hydrolytically labile iodinated species such as WIN 67721, WIN 12901, WIN 68165, and WIN 68209.

The active agent may be formed into particles or nanoparticles by methods known in the art. The methods used to produce the active agent nanoparticles include, but are not limited to, the use of supercritical fluid (SCF) precipitation techniques, particle attrition techniques, and solution precipitation with high agitation. Suitable SCF techniques include, but are not limited to, rapid expansion (RES), solution enhanced diffusion (SEDS), gas-anti solvent (GAS), supercritical antisolvent (SAS), precipitation from gas-saturated solution (PGSS), precipitation with compressed antisolvent (PCA), and aerosol solvent extraction system (ASES). Exemplary methods for the use of SCF precipitation techniques are provided in U.S. patent application Ser. Nos. 10/197,689 and 10/264,030. Suitable particle attrition techniques include, but are not limited to, dry-milling techniques, wet-milling techniques and air-jet milling techniques. Exemplary methods for the use of particle attrition techniques are provided in U.S. Pat. Nos. 6,264,922 and 5,429,824.

The active agent particles produced through the use of these methods can be formulated into aerosol formulations by methods known in the art. In one embodiment the active agent particles are in a nanoparticulate form. Suitable aerosol formulations include, but are not limited to, suspensions, referred to herein as “nanosuspensions”, solutions, referred to herein as “nanosolutions.”

As used in the present disclosure, “nanoparticles” or “nanoparticulate form” refers to particles having an average particle size less than 1 micron (1000 nanomaeters). In one embodiment, the average particle size less than 1000 nanometers. In an alternate embodiment, the average particle size less than 800 nanometers. In yet another alternate embodiment, the average particle size of less than 600 nanometers. The average particle size can be determined using any available particle size measuring techniques, such as, but not limited to measurement of MMAD by cascade impactors. A formulation is considered to have an average particle size if 90% or greater of the particles (determined by weight percentage) in such formulation have such particle size. For example, a drug formulation is deemed to have an average particle size of 600 nanometers if 90% or greater of the particles (determined by weight percentage) in said formulation have an average particle size of less than 600 nanometers. The nanoparticles may be crystalline or amorphous, depending on the method of producing said nanoparticles.

The aerosol formulations described further comprise an excess of a surface tension reducing agent. The aerosol formulation may contain one surface tension reducing agent or more than one surface tension reducing agent. By an excess of surface tension reducing agent, it is meant that a portion of the surface tension reducing agent is free in the aerosol formulation and is not absorbed or otherwise associated with the active agent particles. In one embodiment, at least about 1%, at least about 10%, at least about 25% or at least about 50% of the surface tension reducing agent is free in the aerosol formulation and is not absorbed or otherwise associated with the active agent particles. The purpose of the surface tension reducing agent is to reduce the surface tension of the droplets formed when the aerosol formulation is nebulized/atomized. Suitable surface tension reducing agents include, but are not limited to, include, but are not limited to, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (for example macrogol ethers such as cetomacrogol 1000), polyoxyethylene castor oil derivatives, polyoxyethylene sorbitan fatty acid esters (for example, the commercially available Tweens™ such as polyoxyethylene sorbitan monolaurate, Tween 20, polyoxyethylene sorbitan monoostearate, Tween 60, and polyoxyethylene sorbitan monooleate, Tween 80) polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethylcellulose phthalate, noncrystalline cellulose, magnesium aluminum silicate, triethanolamine, polyvinyl alcohol, and polyvinylpyrrolidone (PVP). Most of these surface tension reducing agents are known compounds for use pharmaceutical formulations and are described in detail in the Handbook of Pharmaceutical Excipients, published jointly by the American Pharmaceutical Association and The Pharmaceutical Society of Great Britain, the Pharmaceutical Press, 1986. In a specific embodiment, the surface tension reducing agent is Tween 20, Tween 60, Tween 80, lecithin or combinations thereof.

A portion of the surface tension reducing agent may be absorbed to the surface of the particles of active agent. As such the surface tension reducing agent may also help maintain the active agent particles in a nanoparticulate form. However, unlike prior art aerosol formulations, there is always an excess of surface tension reducing agent present so that a portion of the surface tension reducing agent is not absorbed or otherwise associated with the active agent particles and can serve to reduce the surface tension of the aerosol formulations described herein.

The aerosol formulation may further comprise other accessory agents as is known in the art.

Although the prior art has been aware of a variety of surface tension reducing agents for use with aerosol formulations, it has been surprisingly found that most of the surface tension reducing agents exert their functionality in the aerosol formulation through strong adsorption to the surface of the active agent particles. Therefore, the surface tension reducing agents are “bound” to the active agent particles and no surface tension reducing agents is freely dispersed or dissolved in the fluid media. Therefore, the active agent particles can be considered a “sink” for the surface tension reducing agents.

While this situation is ideal for active agent particle stabilization, since the surface tension reducing agents are bound to the active agent particles, they are prevented from exerting additional beneficial effects. It is well known that nebulized/atomized droplet formation is enhanced by lowering surface tension. Aerosol formulations comprising pure water and nanoparticulate active agents with strongly adsorbed surface tension reducing agents will exhibit surface tensions above 60 dynes/cm. Reducing the surface tension of the aerosol formulation has been found to significantly improve nebulization/atomization of the aforementioned aerosol formulation into liquid aerosol droplets. In one embodiment, the surface tension of the aerosol formulation is less than that of pure water (nominally 70 dynes/cm). In an alternate embodiment, the surface tension of the aerosol formulation is greater than about 40 dynes/cm and less than about 70 dynes/cm. In yet another alternate embodiment, the surface tension of the aerosol formulation is between about 45 and about 55 dynes/cm. In still a further embodiment, the surface tension of the aerosol formulation is between about 40 dynes/cm and about 60 dynes/cm. Reducing the surface tension allows droplets to form more readily upon mechanical deformation, resulting in a lower average droplet diameter which results in higher respirable fraction. The amount and identity of the surface tension reducing agents can be selected to provide the desired surface tension. Surface tension is measured as is known in the art.

Droplets of an aerosol formulation according to the present disclosure are generated by nebulization typically have average particle sizes in the range of 0.01 microns to 10.0 microns. In one embodiment, average particle sizes are from about 0.01 microns to 6.0. Such particles sizes are considered to have a high respirable fraction and are particularly effective for the pulmonary delivery of therapeutic agents and diagnostic agents.

The surface tension reducing agents can be added in any desired range to produce the surface tension values described above. In one embodiment, the surface tension reducing agent is added from about 0.0001% to 1% w/w based on the final formulation. In an alternate embodiment, the surface tension reducing agent is added from about 0.001% to 0.1% w/w based on the final formulation. In a specific example, polyoxyethylene sorbitan monooleate is added from about 0.001% to 1% w/w based on the final formulation.

The use of surface active agent at levels cited above serve to reduce the surface tension and drive the generation of droplets in ideal particle size ranges (<6 microns). The methods of the present disclosure have excellent application in the aerosolization or nebulization of ‘nanosuspensions.’ for effective liquid aerosol drug delivery to the lung.

The present disclosure also provides for a method for forming an aerosol from said aerosol formulation, said aerosol formulation comprising droplets of an aqueous dispersion of active agent particles, said aqueous dispersion having an excess of a surface tension reducing agent. The active agent particles may be in nanoparticulate form. The active agent may be a therapeutic agent or a diagnostic agent and is insoluble or poorly soluble in water. In one embodiment, the method comprises the steps of:
a) providing an aerosol formulation comprising an aqueous dispersion of active agent particles, said aqueous dispersion having an excess of a surface tension reducing agent; and
b) nebulizing or atomizing said aerosol formulation using an appropriate device to form an aerosol comprising droplets of said aerosol formulation, said droplets comprising particles of said at least one active agent and an excess of the surface tension reducing agent.

The aerosol formulations produced may be nebulized/atomized by a variety of devices such as, but not limited to, jet nebulizers, ultrasonic nebulizers, small orifice aerosol generators, nebulizers, atomizers and vibrating orifice nebulizers. Exemplary nebulizers include, but are not limited to, Aerogen Aeroneb®, Omron MicroAire®, PARI EFlow™, Boeringher Respimat®, Aradigm AERx®, and next generation nebulizers from Repironics, Ventaira, and Profile Therapeutics.

The aerosol formulations may be formulated for use in pressurized inhaler devices, including metered dose inhalers, using propellants such as 1,1,1,2,3,3,-heptafluoro-n-propane and/or 1,1,1,2-tetrafluoroethane or any mixture of both in any proportions. Alternatively the formulations can be spray dried, freeze dried or lyophilized to produce dry powder formulations. The formulations can be packaged into appropriate containers using blow/fill/seal technology as is known in the art.

The present disclosure further provides a method of treating a mammal in need of said treatment, said method comprising the steps of:
a) providing an aerosol formulation comprising an aqueous dispersion of active therapeutic agent particles, said aqueous dispersion having an excess of a surface tension reducing agent;
b) nebulizing or atomizing said aerosol formulation using an appropriate device to form an aerosol comprising droplets of said aerosol formulation, said droplets comprising particles of said at least one active therapeutic agent and an excess of the surface tension reducing agent; and
c) administering said aerosol to the respiratory system of said mammal.

A method for treating using an aerosol formulation of the present disclosure comprises administering to a subject in need of a treatment an effective amount of the of an aerosol composition of the present disclosure comprising at least one therapeutic agent. The subject may be a human or other mammal such as, but not limited to, rabbits, dogs, cats, monkeys, sheep, pigs, horses, bovine animals and the like.

The effective amount for treatment is effective to obtain a desired therapeutic response for a particular therapeutic agent and method of administration. The effective amount therefore, depends upon the particular active agent, the desired therapeutic effect, the route of administration, the desired duration of treatment, the disease state or condition being treated and other factors.

The aerosol formulations may be used to treat a variety of diseases or conditions depending on the nature of the active agent. In one embodiment, when the active agent is budesonide, the aerosol formulations may be used to treat a variety of respiratory diseases, such as but not limited to, asthma, chronic obstructive pulmonary disease, emphysema, respiratory distress syndrome, chronic bronchitis, and cystic fibrosis.

The present disclosure still further provides a method of diagnostic imaging of a mammal in need of such diagnosis, said method comprising:
a) providing an aerosol formulation comprising an aqueous dispersion of active diagnostic agent particles, said aqueous dispersion having an excess of a surface tension reducing agent;
b) nebulizing or atomizing said aerosol formulation using an appropriate device to form an aerosol comprising droplets of said aerosol formulation, said droplets comprising particles of said at least one active diagnostic agent and an excess of the surface tension reducing agent;
c) administering said aerosol to the respiratory system of said mammal; and
d) imaging said active diagnostic agent in said respiratory system.

A method for diagnostic imaging for use in medical procedures using an aerosol formulation of the present disclosure comprises administering to a subject in need of a diagnostic image an effective amount of the of an aerosol composition of the present disclosure comprising at least one diagnostic agent. The subject may be a human or other mammal such as, but not limited to, rabbits, dogs, cats, monkeys, sheep, pigs, horses, bovine animals and the like.

After administration of the diagnostic agent to the subject, the subject mammal is maintained for a time period sufficient for the administered diagnostic agent to be distributed throughout the subject and enter the tissues of the subject. Typically, a sufficient time period is from about 20 minutes to about 90 minutes and. Once the diagnostic agent is distributed, at least a portion of the body of the subject containing the administered diagnostic agent is exposed to a diagnostic procedure to produce an image corresponding to the presence of the diagnostic agent. For example the subject may be exposed to x-rays to produce an x-ray pattern corresponding to the presence of the diagnostic agent or to a magnetic field to produce a magnetic resonance image pattern corresponding to the presence of the diagnostic agent. The image pattern can then be visualized by techniques well known in the art.

Any x-ray visualization technique, preferably, a high contrast technique such as computed tomography, can be applied in a conventional manner. Alternatively, the image pattern can be observed directly on an x-ray sensitive phosphor screen-silver halide photographic film combination or by use of a storage phosphor screen. Visualization with a magnetic resonance imaging system can be accomplished with commercially available magnetic imaging systems. Commercially available magnetic resonance imaging systems may be used in a conventional manner.

A contrast effective amount of the diagnostic agent containing composition is that amount necessary to provide tissue visualization with a diagnostic method, for example, magnetic resonance imaging or x-ray imaging. Means for determining a contrast effective amount in a particular subject will depend, as is well known in the art, on the nature of the diagnostic agent being used, the mass of the subject, the sensitivity of the diagnostic method and the like.

### EXAMPLE 1

The present example provides several exemplary embodiments of aerosol formulations according to the present disclosure with budesonide as the active agent and polyoxyethylene sorbitan monooleate (Tween 80) as the surface tension reducing agent. In this example, the budesonide particles are initially processed to produce a sterile bulk drug intermediate solution which is further processed into the final aerosol formulation.

In the initial processing, the crystalline budesonide starting material subject to a milling step to reduce the size of the budesonide particles. The milling step is accomplished by milling the crystalline budesonide starting material in a dilute solution of Tween 80 and a milling media to a substantially smaller diameter. The budesonide particles produced are stabilized by the subsequent addition of hydrogenated soy lecithin (which is heated above its phase transition temperature for proper dispersal) and disodium edetate. The resulting concentrated bulk drug intermediate suspension is autoclaved in pressure vessels to achieve sterilization. The resulting bulk drug intermediate solution comprises hydrogenated soy lecithin, Tween 80 and disodium edetate.

The bulk drug intermediate solution is further processing into a desired aerosol formulation according to the present disclosure. The bulk drug intermediate solution is diluted aseptically to the appropriate strength by addition to a citrate-buffered isotonic saline solution which is sterilized by terminal filtration prior to filling into unit dose vials. The final pH of the aerosol formulation is from about pH 4 to about pH 7. One of ordinary skill in the art can alter the sodium citrate and citric acid ratios to produce the desired pH. The budesonide particles in the final aerosol formulation have an average particle size in the range of 300 to 600 nanometers. The submicron particle size of the budesonide particles facilitates the distribution of the budesonide particles into the smaller, more readily respirable droplets produced by nebulization/atomization of the novel aerosol formulation of the present disclosure. Final unit doses are packaged in LDPE (Rexene®) vials and 4 unit dose vials are overwrapped with foil to protect from light and moisture loss.

In the final aerosol formulation, the ratio of budesonide to Tween 80 ranges from about 0.5: 1 to about 15:1, while the ratio of budesonide to lecithin ranges from about 15:1 to about 20:1. In one embodiment, the ratio of budesonide to Tween 80 is from about 1.5:1 to about 12.5:1 and the ratio of budesonide to lecithin is about 20:1.

TABLE 1
Exemplary Aerosol Formulations in 2.0 ml Unit Dose Volumes

| | Formulation 1 | | Formulation 2 | | Formulation 3 | | Formulation 4 | |
|---|---|---|---|---|---|---|---|---|
| Budesonide | 0.0625 | mg | 0.125 | mg | 0.250 | mg | 0.50 | mg |
| Lecithin | 0.0031 | mg | 0.0062 | mg | 0.0125 | mg | 0.025 | mg |
| Tween 80 | 0.04 | mg | 0.04 | mg | 0.04 | mg | 0.04 | mg |
| Sodium Chloride | 17.0 | mg | 17.0 | mg | 17.0 | mg | 17.0 | mg |
| Sodium Citrate Dihydrate | 0.69 | mg | 0.69 | mg | 0.69 | mg | 0.69 | mg |
| Citric Acid | 0.74 | mg | 0.74 | mg | 0.74 | mg | 0.74 | mg |
| Edetate disodium | 0.10 | mg | 0.10 | mg | 0.10 | mg | 0.10 | mg |
| Water for injection | q.s. ad 2.0 | ml | q.s. ad 2.0 | ml | q.s. ad 2.0 | ml | q.s. ad 2.0 | ml |

TABLE 2
Exemplary Aerosol Formulations in 1.5 ml Unit Dose Volumes

| | Formulation 1 | | Formulation 2 | |
|---|---|---|---|---|
| Budesonide | 0.125 | mg | 0.250 | mg |
| Lecithin | 0.0071 | mg | 0.0132 | mg |
| Tween 80 | 0.0371 | mg | 0.0432 | mg |
| Sodium Chloride | 12.75 | mg | 12.75 | mg |
| Sodium Citrate Dihydrate | 0.936 | mg | 0.936 | mg |
| Citric Acid | 0.282 | mg | 0.282 | mg |
| Edetate disodium | 0.075 | mg | 0.075 | mg |
| Water for injection | q.s. ad 1.5 | ml | q.s. ad 1.5 | ml |

The aerosol formulation may be used as described in the present disclosure.

The foregoing description describes the methods of the present disclosure. Additionally, the disclosure describes only certain embodiments of the compounds but it is to be understood that the teachings of the present disclosure are capable of use in various other combinations, modifications, and environments and is capable of change or modification within the scope of the inventive concept as expressed herein, commensurate with the above teachings and/or the skill or knowledge of the relevant art. The embodiments described hereinabove are further intended to explain best modes known of practicing the invention and to enable others skilled in the art to utilize the invention in such, or other, embodiments and with the various modifications required by the particular applications or uses of the invention. Accordingly, the description is not intended to limit the invention to the form disclosed herein. All references cited herein are incorporated by reference as if fully set forth in this disclosure.

What is claimed:

1. An aerosol formulation for respiratory delivery, said aerosol formulation comprising an aqueous dispersion of at least one active agent said aqueous dispersion having an excess of at least one surface tension reducing agent to reduce a surface tension of the aerosol formulation and said active agent being present in a particle form.

2. The formulation of claim 1 where said surface tension reducing agent is selected from the group consisting of gelatin, casein, lecithin, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycol, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethylcellulose phthalate, noncrystalline cellulose, magnesium aluminum silicate, triethanolamine, polyvinyl alcohol, and polyvinylpyrrolidone

3. The formulation of claim 1 where said surface tension reducing agent is selected from the group consisting of polyoxyethylene sorbitan monolaurate, polyoxyethylene sorbitan monoostearate, and polyoxyethylene sorbitan monooleate.

4. The formulation of claim 1 where at least about 1%, at least about 10%, at least about 25% or at least about 50% of the surface tension reducing agent is free in the aerosol formulation and is not absorbed on the surface of the particles of the active agent.

5. The formulation of claim 1 where said surface tension is less than 70 dynes/cm, less than 60 dynes/cm, less than 50 dynes/cm or less than 40 dynes/cm.

6. The formulation of claim 1 where said surface tension is between about 40 dynes/cm and about 70 dynes/cm, between about 45 and about 55 dynes/cm or between about 40 dynes/cm and about 60 dynes/cm.

7. The formulation of claim 1 where said surface tension reducing agent is present from about 0.0001% to about 1% (w/w) in said aerosol formulation.

8. The formulation of claim 1 where an aerosol droplet produced from said formulation has a size less than about 10 microns, less than about 6 microns or less than about 1 micron.

9. The formulation of claim 1 where said active agent particles are in nanoparticulate form.

10. The formulation of claim 1 where said active agent particles have an average particle size less than 1000 nanometers, less than 800 nanometers or less than 600 nanometers.

11. The formulation of claim 1 where said active agent is insoluble in an aqueous media.

12. The formulation of claim 1 where said active agent has a solubility in an aqueous media of less than about 10 mg/ml or less than about 1 mg/ml.

13. The formulation of claim 1 where said active agent is a therapeutic agent or a diagnostic agent.

14. The formulation of claim 13 where said therapeutic agent is selected from the group consisting of, analgesic agents, anti-inflammatory agents, anthelmintic agents, anti-arrhythmic agents, antibiotics agents, anticoagulant agents, antidepressant agents, antidiabetic agents, antiepileptic agents, antihistamine agents, antihypertensive agents, antimuscarinic agents, antimycobacterial agents, antineoplastic agents, immunosuppressant agents, antithyroid agents, antiviral agents, anxiolytic sedatives, astringent agents, beta-adrenoceptor blocking agents, blood products, blood substitutes, cardiac inotropic agents, corticosteroid agents, antibodies, cough suppressants, diuretic agents, dopaminergic agents, haemostatic agents, immunological agents, lipid regulating agents, muscle relaxants, parasympathomimetic agents, parathyroid, calcitonin, prostaglandins, sex hormones, anti-allergic agents, stimulants, anoretics, sympathomimetics, thyroid agents, vasodilators and xanthines.

15. The formulation of claim 13 where said therapeutic agent is a corticosteroid.

16. The formulation of claim 13 where said active agent is selected from the group consisting of budesonide, dexamethasone, cortisone, prednisone, methylprednisone, hydrocortisone, beclomethasone dipropionate, betamethasone, flunisolide, fluticasone, flumethasone, fludrocortisone, diflorasone diacetate, flunisolide, fluocinolone acetonide, fluocinonide, fluorometholone, flurandrenolide, fluprednisolone, methylprednisone, paramethasone, prednisone, prednisolone, triamcinolone, alclometasone, amcinonide, cortisone, tetrahydrocortisol, clobetasol, ciclesonide, desonide, desiximetasonedeflazacort, halcinonide, medrysone, mometasone, paramethasone, tipredane, triamcinolone, rofleponide, aldosterone, fludrocortisone, desoxycortiscosterone acetate and the esters, acetals, or salts of the foregoing.

17. The formulation of claim 13 where said therapeutic agent is budesonide.

18. The formulation of claim 13 where said diagnostic agent is selected from the group consisting of WIN-8883 (ethyl 3,5-diacetamido-2,4,6triiodobenzoate), WIN 67722 (6-ethoxy-6-oxohexyl-3,5-bis(acetamido)-2,4,6-triiodobenzoate), WIN 16318 (ethyl-2-(3,5-bis(acetamido)-2,4,6-triiodobenzoyloxy)butyrate), WIN 12901 (ethyl diatrizoxyacetate), WIN 16923 (ethyl 2-(3,5bis(acetamido)-2,4,6-triiodobenzoyloxy)propionate), WIN 65312 (N-ethyl 2-(3,5-bis(acetamido)-2,4,6triiodobenzoyloxy acetamide), WIN 12855 (isopropyl 2(3,5-bis(acetamido)-2,4,6-triiodobenzoyloxy)acetamide), WIN 67721 (diethyl 2-(3,5-bis(acetamido)-2,4,6triiodobenzoyloxy malonate); WIN 67585 (ethyl 2-(3,5bis(acetamido)-2,4,6-triiodobenzoyloxy)phenylacetate); propanedioic acid, WIN 68165 ([[3,5-bis(acetylamino)2,4,5-triodobenzoyl]oxy]-,bis(1-methyl)ester), WIN 68209 (3,5-bis(acetylamino)-2,4,6triodo-,4-(ethyl-3-ethoxy-2-butenoate)ester) and benzoic acid.

19. An aerosol formulation for respiratory delivery, said aerosol formulation comprising an aqueous dispersion of nanoparticulate budesonide and an excess of polyoxyethylene sorbitan monooleate as a surface tension reducing agent and lecithin.

20. The aerosol formulation of claim 19 where said nanoparticulate budesonide has an average particle size less than 1000 nanometers, less than 800 nanometers or less than 600 nanometers.

21. The formulation of claim 19 where an aerosol droplet produced from said formulation has a size less than about 10 microns, less than about 6 microns or less than about 1 micron.

22. The aerosol formulation of claim 19 where said pH is between about 4 to about 7.

23. The formulation of claim 19 where said budesonide is present in an amount between about 0.03125 mg/ml and about 0.250 mg/ml, said budesonide is present in a ratio of about 0.5:1 to about 15:1 with respect to said polyoxyethylene sorbitan monooleate in a ratio of about 15:1 to about 20:1 with respect to said lecithin and said formulation has a pH between about 4 and about 7.

24. The aerosol formulation of claim 23 where said nanoparticulate budesonide has an average particle size less than 1000 nanometers, less than 800 nanometers or less than 600 nanometers.

25. The formulation of claim 24 where an aerosol droplet produced from said formulation has a size less than about 10 microns, less than about 6 microns or less than about 1 micron.

26. The formulation of claim 19 where said budesonide is present in an amount between about 0.03125 mg/ml and about 0.250 mg/ml, said budesonide is present in a ratio of about 1.5:1 to about 12.5:1 with respect to said polyoxyethylene sorbitan monooleate in a ratio of about 20:1 with respect to said lecithin and said formulation has a pH between about 4 and about 7.

27. The aerosol formulation of claim 26 where said nanoparticulate budesonide has an average particle size less than 1000 nanometers, less than 800 nanometers or less than 600 nanometers.

28. The formulation of claim 27 where an aerosol droplet produced from said formulation has a size less than about 10 microns, less than about 6 microns or less than about 1 micron.
